SEQUENCE LISTING (31 sequences)

SEQ ID NO: 1 - protein, 6 aa, Spirulina sp.
LNRTFE

SEQ ID NO: 2 - protein, 6 aa, Spirulina sp.
GELRVR

SEQ ID NO: 3 - protein, 6 aa, Spirulina sp.
RLLKEA

SEQ ID NO: 4 - protein, 6 aa, Spirulina sp.
LPTKEE

SEQ ID NO: 5 - protein, 6 aa, Spirulina sp.
SNKRLD

SEQ ID NO: 6 - protein, 6 aa, Spirulina sp.
MTCEDR

SEQ ID NO: 7 - protein, 6 aa, Spirulina sp.
DTRGEM

SEQ ID NO: 8 - protein, 5 aa, Spirulina sp.
PYTTQ

SEQ ID NO: 9 - protein, 6 aa, Spirulina sp.
LDRFRQ

SEQ ID NO: 10 - protein, 6 aa, Spirulina sp.
TPRYNE

SEQ ID NO: 11 - protein, 6 aa, Spirulina sp.
VKQLEE

SEQ ID NO: 12 - protein, 6 aa, Spirulina sp.
NDLYRL

SEQ ID NO: 13 - protein, 5 aa, Spirulina sp.
TPLEE

SEQ ID NO: 14 - protein, 5 aa, Spirulina sp.
LRFRQ

SEQ ID NO: 15 - protein, 5 aa, Spirulina sp.
ERRYP

SEQ ID NO: 16 - protein, 6 aa, Spirulina sp.
DREVLN

SEQ ID NO: 17 - protein, 6 aa, Spirulina sp.
FPADKK

SEQ ID NO: 18 - protein, 10 aa, synthetic construct
PGIIGLPGPA

SEQ ID NO: 19 - protein, 8 aa, synthetic construct
AVGPVGPS

SEQ ID NO: 20 - protein, 8 aa, synthetic construct
ERGPPGPP

SEQ ID NO: 21 - protein, 7 aa, synthetic construct
APGELPY

SEQ ID NO: 22 - protein, 6 aa, synthetic construct
DIFDPL

SEQ ID NO: 23 - protein, 6 aa, synthetic construct
LDVAPL

SEQ ID NO: 24 - protein, 4 aa, synthetic construct
ICRD

SEQ ID NO: 25 - protein, 5 aa, synthetic construct
LCGEC

SEQ ID NO: 26 - DNA, 20 bp, synthetic construct
  source 1..20: mol_type = other DNA
tgggtcaaatacgactgccc

SEQ ID NO: 27 - DNA, 20 bp, synthetic construct
  source 1..20: mol_type = other DNA
tggctcatatctctccacgc

SEQ ID NO: 28 - DNA, 20 bp, synthetic construct
  source 1..20: mol_type = other DNA
cgcgagtacaaccttcttgc

SEQ ID NO: 29 - DNA, 20 bp, synthetic construct
  source 1..20: mol_type = other DNA
cgtcatccatggcgaactgg

SEQ ID NO: 30 - DNA, 20 bp, synthetic construct
  source 1..20: mol_type = other DNA
actcctacgggaggcagcag

SEQ ID NO: 31 - DNA, 20 bp, synthetic construct
  source 1..20: mol_type = other DNA
ggactachvgggtwtctaat

(19) United States
(12) Patent Application Publication
(10) Pub. No.: US 2026/0125424 A1
(43) Pub. Date: May 7, 2026

(54) BIOACTIVE PEPTIDE PCT-1, AND PREPARATION METHOD AND APPLICATION THEREOF

(71) Applicants: YANTAI INST COASTAL ZONE RES CAS, Yantai (CN); YANTAI ZHIGONG BIOMEDICAL TECH CO LTD, Yantai (CN)

(72) Inventors: Wenjun LI, Yantai (CN); Tuanjie CHE, Yantai (CN); Song QIN, Yantai (CN); Chenyang LU, Yantai (CN); Jinzhang XU, Yantai (CN); Libo YAN, Yantai (CN); Guizhen XIE, Yantai (CN); Ying KAN, Yantai (CN)

(73) Assignees: YANTAI INST COASTAL ZONE RES CAS, Yantai (CN); YANTAI ZHIGONG BIOMEDICAL TECH CO LTD, Yantai (CN)

(21) Appl. No.: 19/435,866

(22) Filed: Dec. 30, 2025

Related U.S. Application Data
(63) Continuation of application No. PCT/CN2024/130286, filed on Nov. 6, 2024.

(30) Foreign Application Priority Data
Oct. 29, 2024 (CN) 202411514023.4

(51) Int. Cl. C07K 7/06; A61K 38/00; A61P 1/14; A61P 39/06

(52) U.S. Cl. CPC C07K 7/06; A61P 1/14; A61P 39/06; A61K 38/00

(57) ABSTRACT

A bioactive peptide PCT-1, and preparation method and application thereof are provided. The amino acid sequence of the bioactive peptide PCT-1 is LNRTFE, as shown in SEQ ID NO: 1, and the bioactive peptide PCT-1 has antioxidant and intestinal flora-regulating functions. Extracted from spirulina protein peptides, the bioactive peptide PCT-1 can significantly increase the activities of SOD and GSH-Px in serum, and significantly reduce the content of MDA in serum; its in vivo antioxidant activity is comparable to that of the positive control Trolox. Meanwhile, compared with the blank control group, the bioactive peptide PCT-1 can also reduce the α-diversity index of intestinal flora, change the overall structure of intestinal flora, regulate the abundance of intestinal flora at various taxonomic levels, and significantly increase the contents of acetic acid and butyric acid. Therefore, the bioactive peptide PCT-1 can be applied in functional foods for antioxidation and intestinal flora regulation.

### CROSS REFERENCE TO THE RELATED APPLICATIONS

This application is a continuation application of International Application No. PCT/CN2024/130286, filed on Nov. 6, 2024, which is based upon and claims priority to Chinese Patent Application No. 202411514023.4, filed on Oct. 29, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBYTZX010-PKG_Sequence_Listing.xml, created on Nov. 25, 2025, and is 27,624 bytes in size.

### TECHNICAL FIELD

The present invention relates to a small-molecule peptide, and preparation method and application thereof, and in particular to a small-molecule bioactive peptide PCT-1 with antioxidant and intestinal flora-regulating functions, as well as its preparation method and application in functional foods for antioxidation and intestinal flora regulation. The present invention belongs to the field of biotechnology.

### BACKGROUND

The occurrence of various diseases such as obesity, aging, cancer, cardiovascular diseases, and Alzheimer's disease has been proven to be closely related to the excessive production of free radicals. The human body normally can regulate oxidative balance through a variety of antioxidant mechanisms, including scavenging free radicals to block the chain reaction of free radicals, chelating metal ions to inhibit the production of free radicals, and regulating endogenous glutathione peroxidase (GSH-Px), superoxide dismutase (SOD), and catalase (CAT) to scavenge free radicals. When the endogenous oxidative balance is disrupted, the body needs exogenous supplementation of antioxidants. Traditional antioxidants are generally derived from chemical synthesis and have shortcomings such as poor stability and strong toxic and side effects. In contrast, antioxidant peptides derived from protein hydrolysis have advantages such as safety, non-toxicity, and strong stability, which are more in line with consumer requirements.

The human intestine contains approximately 1,000 species and 100 trillion microorganisms, mainly including bacteria, yeasts, and parasites. The number of genes carried by these intestinal microorganisms is more than 100 times that of human genes, and they are known as the “invisible organ”. Imbalance of intestinal flora is closely related to the occurrence of obesity, hypertension, intestinal inflammation, cardiovascular diseases, etc. Moreover, helping to regulate intestinal flora is also one of the functions that health food is allowed to claim in the Catalogue of Health Functions Allowed for Health Food Claims—Non-Nutrient Supplements (2023 Edition). In addition to the direct supplementation of probiotics, bioactive peptides have also been reported to have the function of regulating intestinal flora.

In the graduation thesis Effects of Isolation and Purification Process on the Structure and Activity of Antioxidant Peptides from Tilapia Skin, Zuo Yijin described in detail the antioxidant activity of the enzymatic hydrolysate of tilapia skin, as well as the core functional antioxidant peptide components therein, such as the decapeptide PGIIGLPGPA (SEQ ID NO: 18), the octapeptide AVGPVGPS (SEQ ID NO: 19), and the octapeptide ERGPPGPP (SEQ ID NO: 20). In the graduation thesis Isolation, Identification and Functional Verification of Bioactive Peptides from Antarctic Krill, Wang Yiju described the process of preparing antioxidant peptides from Antarctic krill, proved that components with strong antioxidant activity are mainly enriched in low-molecular-weight polypeptides, and identified that the heptapeptide APGELPY (SEQ ID NO: 21), the hexapeptide DIFDPL (SEQ ID NO: 22), and the hexapeptide LDVAPL (SEQ ID NO: 23) have good antioxidant effects. In Isolation and Identification of Antioxidant Peptides Derived from Ovalipes punctatus Waste Containing Meat Scraps, Yu Hui described the evaluation of the antioxidant activity of the trypsin enzymatic hydrolysate of the Ovalipes punctatus waste containing meat scraps, and isolated two antioxidant peptides with strong ABTS+ scavenging ability, namely the tripeptide YEG and the dipeptide YE. In addition, Feng Ziqi et al. described the regulatory effect of bioactive peptides from Mytilus coruscus on the intestinal flora of mice with alcohol-induced liver injury in the article Protective Effects of Bioactive Peptides from Mytilus coruscus on Alcohol induced Liver Injury and Their Regulation of Gut Microbiota in Mice; in the graduation thesis Study on the Effect of Giant Salamander Bioactive Peptides on Intestinal Flora, Chen Xinai specifically studied the regulatory effect of giant salamander bioactive peptides on the intestinal flora structure and metabolism of obese mice induced by a high-fat diet. In the graduation thesis Preparation and Activity Study of Collagen Peptides from Micropterus salmoides Skin, Han Mengyao mentioned the regulatory effect of collagen peptides from Micropterus salmoides skin on the intestinal flora of immunocompromised mice. Furthermore, Jiaojiao Han et al. described the intestinal flora-regulating effect of the antioxidant peptides tetrapeptide ICRD (SEQ ID NO: 24) and pentapeptide LCGEC (SEQ ID NO: 25) in the article The novel peptides ICRD and LCGEC screened from tuna roe show antioxidative activity via Keap1/Nrf2-ARE pathway regulation and gut microbiota modulation. From the structures of bioactive peptides disclosed in the above literatures, it can be found that the structures of bioactive peptides with antioxidant and intestinal flora-regulating functions are diverse, and there is no obvious sequence structure rule.

### SUMMARY

The objective of the present invention is to provide a small-molecule bioactive peptide that is screened from spirulina protein peptides, has antioxidant and intestinal flora-regulating functions, and can be applied in functional foods for antioxidation and intestinal flora regulation.

To achieve the above objective, the present invention adopts the following technical solutions:

A bioactive peptide PCT-1, where the amino acid sequence of the bioactive peptide PCT-1 is LNRTFE, as shown in SEQ ID NO: 1 in the sequence listing, and the bioactive peptide PCT-1 has antioxidant and intestinal flora-regulating functions.

An application of the bioactive peptide PCT-1 in functional foods for antioxidation and intestinal flora regulation.

A preparation method of the bioactive peptide PCT-1, which adopts a solid-phase synthesis method, specifically:

using Fmoc-protected amino acids as raw materials and polystyrene resin as a solid-phase carrier, and performing solid-phase synthesis by adopting an Fmoc solid-phase synthesis strategy.

Another preparation method of the bioactive peptide PCT-1, which adopts an enzymatic hydrolysis method, specifically:
(1) taking spirulina protein, adding water with a mass 9 times that of the spirulina protein, and adding a compound protease at an enzyme-to-substrate ratio of 0.5% for enzymatic hydrolysis; conducting the enzymatic hydrolysis at a temperature of 55° C. and a pH value of 8.0 for 6 h to obtain an enzymatic hydrolysate, where the compound protease is formed by mixing alkaline protease and neutral protease at a mass ratio of 1:1;
(2) after the enzymatic hydrolysis is completed, filtering the enzymatic hydrolysate with eight layers of gauze to remove residues, so as to obtain a spirulina protein peptide enzymatic hydrolysate; and
(3) freeze-drying the spirulina protein peptide enzymatic hydrolysate to obtain a spirulina protein peptide powder, where the spirulina protein peptide powder contains a relatively large amount of the bioactive peptide PCT-1.

The advantages of the present invention are as follows: the bioactive peptide PCT-1 screened from spirulina protein peptide in the present invention can significantly increase the activity of SOD and the activity of GSH-Px in serum, and significantly reduce the content of malondialdehyde (MDA) in serum, and its in vivo antioxidant activity is equivalent to that of the positive control Trolox; at the same time, compared with the blank control group, the PCT-1 treatment can reduce the α diversity index of intestinal flora, change the overall structure of intestinal flora (β diversity index), regulate the abundance of intestinal flora at various taxonomic levels, and significantly increase the contents of acetic acid and butyric acid; the bioactive peptide PCT-1 can be applied in functional foods for antioxidation and intestinal flora regulation.

### BRIEF DESCRIPTION OF THE DRAWINGS

FIG. 1 is a schematic diagram showing the binding mode between the bioactive peptide PCT-1 and Keap1;

FIG. 2 is a schematic diagram of the local magnification of FIG. 1;

FIGS. 3A-3C are diagrams showing the effect of bioactive peptide PCT-1 treatment on the physiological indicators of mice, where FIG. 3A is the diagram of the effect on the body weight gain of mice, FIG. 3B is the diagram of the effect on the spleen index of mice, FIG. 3C is the diagram of the effect on the liver index of mice, and * indicates P<0.05, ** indicates P<0.01;

FIGS. 4A-4D are diagrams showing the effect of bioactive peptide PCT-1 treatment on the serum antioxidant indicators of mice, where FIG. 4A is the diagram of the effect on the serum MDA content of mice, FIG. 4B is the diagram of the effect on the serum SOD activity of mice, FIG. 4C is the diagram of the effect on the serum CAT activity of mice, FIG. 4D is the diagram of the effect on the serum GSH-Px activity of mice, and * indicates P<0.05, ** indicates P<0.01;

FIGS. 5A-5B are diagrams showing the effect of bioactive peptide PCT-1 treatment on the transcriptional level of the keap1 gene in mice, where FIG. 5A is the diagram of the effect on the transcriptional level of the keap1 gene in the mouse brain, FIG. 5B is the diagram of the effect on the transcriptional level of the keap1 gene in the mouse liver, and * indicates P<0.05, ** indicates P<0.01;

FIG. 6 is a diagram showing the effect of bioactive peptide PCT-1 treatment on the β diversity of intestinal flora in mice;

FIGS. 7A-7C are diagrams showing the effect of bioactive peptide PCT-1 treatment on the content of short-chain fatty acids in the intestine of mice, where FIG. 7A is the diagram of the effect on the acetic acid content in the mouse intestine, FIG. 7B is the diagram of the effect on the propionic acid content in the mouse intestine, FIG. 7C is the diagram of the effect on the butyric acid content in the mouse intestine, and ** indicates P<0.01, *** indicates P<0.001.

### DETAILED DESCRIPTION OF THE EMBODIMENTS

The present invention is specifically described below in conjunction with the drawings and specific examples.

### I. Preparation of Spirulina Protein Peptide Samples

Spirulina was sequentially cleaned, dried, crushed, and sieved to obtain spirulina powder. The spirulina powder was added to a 0.01 M phosphate buffer solution (pH 6.8) at a ratio of 1 kg:10 L, fully mixed in a granule mixer, then cyclically ground using a colloid mill. After that, the mixture with broken cell walls was centrifuged to remove solid residues, and the supernatant was filtered sequentially through a filter cloth with a pore size of 25 μm and a filter membrane with a pore size of 0.45 μm to obtain a crude spirulina protein extract. The crude spirulina protein extract was subjected to ultrafiltration concentration using ultrafiltration membranes with molecular weight cut-offs of 5 kDa and 10 kDa respectively; the ultrafiltration fraction with a molecular weight in the range of 5 kDa-10 kDa was collected and spray-dried to obtain spirulina protein.

Spirulina protein was taken, and water with a mass 9 times that of the spirulina protein was added (the mass concentration of spirulina protein is 10%); then, a compound protease (formed by mixing alkaline protease and neutral protease at a mass ratio of 1:1) was added at an enzyme-to-substrate ratio of 0.5% for enzymatic hydrolysis. The enzymatic hydrolysis was conducted at a temperature of 55° C. and a pH value of 8.0 for 6 h.

After the enzymatic hydrolysis was completed, the enzymatic hydrolysate was filtered with eight layers of gauze to remove residues, thereby obtaining the spirulina protein peptide enzymatic hydrolysate.

The spirulina protein peptide enzymatic hydrolysate was freeze-dried to obtain spirulina protein peptide powder.

### II. Acquisition of Polypeptide Sequences in Spirulina Protein Peptides

The spirulina protein peptide obtained above was determined using liquid chromatography-tandem mass spectrometry (LC-MS/MS), and the determination results were analyzed using mass spectrometry analysis software to obtain several polypeptide sequences.

The LC-MS/MS determination conditions are as follows:
(1) In the liquid phase method: The chromatographic column was C18 (3 μm, 250 mm×75 μm, Eksigent); Phase A was water containing 0.1% formic acid, and Phase B was acetonitrile containing 0.1% formic acid. The flow rate was 300 nL/min, the injection volume was 4 μL, and a 60-minute chromatographic gradient was used. The specific elution gradient was: 0-48 min, Phase A decreased uniformly from 95% to 60%; 48-55 min, Phase A decreased uniformly from 60% to 30%; 55-56 min, Phase A decreased uniformly from 30% to 0%; 56-60 min, Phase A was maintained at 0%.
(2) In the mass spectrometry method: Orbitrap Exploris 480 (Thermofisher) was used, with positive ion detection mode. The primary resolution was 120000, the AGC was set to 300, and the scanning range was 200-1600 m/z. The MIPS mode was set to “peptide”, the selected charge states were 1-5, the secondary resolution was 15000, and the isolation window was 1.6 m/z.
   III. Screening of Bioactive Peptides with Peak Area ≥2×10⁷ and Amino Acid Count ≤6

From the several polypeptide sequences obtained above, 17 bioactive peptides with a peak area of ≥2×10⁷ and an amino acid count of ≤6 were finally screened out. The specific screening results are shown in Table 1.

TABLE 1
Sequences of bioactive peptides with peak area ≥2 × 10⁷ and amino
acid count ≤6 in spirulina protein peptides

| | | Peak area | Molecular |
|---|---|---|---|
| No. | Sequence of bioactive peptide | (10⁷) | weight (Da) |
| 1 | LNRTFE (SEQ ID NO: 1) | 44.32 | 778.3973 |
| 2 | GELRVR (SEQ ID NO: 2) | 31.96 | 728.4293 |
| 3 | RLLKEA (SEQ ID NO: 3) | 15.56 | 728.4545 |
| 4 | LPTKEE (SEQ ID NO: 4) | 7.35 | 715.3752 |
| 5 | SNKRLD (SEQ ID NO: 5) | 6.33 | 731.3926 |
| 6 | MTCEDR (SEQ ID NO: 6) | 6.12 | 753.2786 |
| 7 | DTRGEM (SEQ ID NO: 7) | 5.74 | 707.2908 |
| 8 | PYTTQ (SEQ ID NO: 8) | 4.16 | 608.2806 |
| 9 | LDRFRQ (SEQ ID NO: 9) | 3.30 | 833.4507 |
| 10 | TPRYNE (SEQ ID NO: 10) | 3.30 | 778.361 |
| 11 | VKQLEE (SEQ ID NO: 11) | 2.97 | 744.4017 |
| 12 | NDLYRL (SEQ ID NO: 12) | 2.94 | 792.413 |
| 13 | TPLEE (SEQ ID NO: 13) | 2.70 | 587.2802 |
| 14 | LRFRQ (SEQ ID NO: 14) | 2.35 | 718.4238 |
| 15 | ERRYP (SEQ ID NO: 15) | 2.23 | 719.3715 |
| 16 | DREVLN (SEQ ID NO: 16) | 2.22 | 744.3766 |
| 17 | FPADKK (SEQ ID NO: 17) | 2.16 | 704.3857 |

IV. Screening of Bioactive Peptides with Strong Binding Ability to Keap1

The 17 polypeptide sequences in Table 1 were separately subjected to molecular docking with Keap1 using Discovery Studio software. Before docking, the 2D structures of the polypeptides were converted to 3D structures through energy minimization, and polypeptide sequences with strong binding ability to Keap1 were screened out. The 3D structure of the Keap1 protein can be downloaded from the RCSB Protein Data Bank (PDB ID: 4IFJ). The docking results are represented by the docking score (-CiE); the larger the -CiE value, the stronger the interaction between the polypeptide and Keap1.

The molecular docking results of these 17 polypeptide sequences are shown in Table 2.

TABLE 2
Predicted results of the interaction between
spirulina protein peptide components and Keap1

| | | -CiE |
|---|---|---|
| No. | Sequence of bioactive peptide | (kcal/mol) |
| 1 | LNRTFE (SEQ ID NO: 1) | 82.7197 |
| 2 | GELRVR (SEQ ID NO: 2) | 75.9773 |
| 3 | RLLKEA (SEQ ID NO: 3) | 71.7847 |
| 4 | LPTKEE (SEQ ID NO: 4) | 78.5337 |
| 5 | SNKRLD (SEQ ID NO: 5) | 69.78 |
| 6 | MTCEDR (SEQ ID NO: 6) | 88.7351 |
| 7 | DTRGEM (SEQ ID NO: 7) | 79.1091 |
| 8 | PYTTQ (SEQ ID NO: 8) | 69.9412 |
| 9 | LDRFRQ (SEQ ID NO: 9) | 79.4809 |
| 10 | TPRYNE (SEQ ID NO: 10) | 87.2041 |
| 11 | VKQLEE (SEQ ID NO: 11) | 82.692 |
| 12 | NDLYRL (SEQ ID NO: 12) | 91.1057 |
| 13 | TPLEE (SEQ ID NO: 13) | 86.5239 |
| 14 | LRFRQ (SEQ ID NO: 14) | 59.7072 |
| 15 | ERRYP (SEQ ID NO: 15) | 74.4941 |
| 16 | DREVLN (SEQ ID NO: 16) | 94.6345 |
| 17 | FPADKK (SEQ ID NO: 17) | 67.4443 |

### V. Molecular Docking Analysis

Among the bioactive peptides with a peak area ≥10×10⁷, the hexapeptide LNRTFE (designated as PCT-1, SEQ ID NO: 1) exhibited the largest-CiE value (82.7197 kcal/mol) in docking with Keap1, and its peak area (44.32×10⁷) ranked first. Therefore, the hexapeptide LNRTFE (SEQ ID NO: 1) was selected for further predictive analysis.

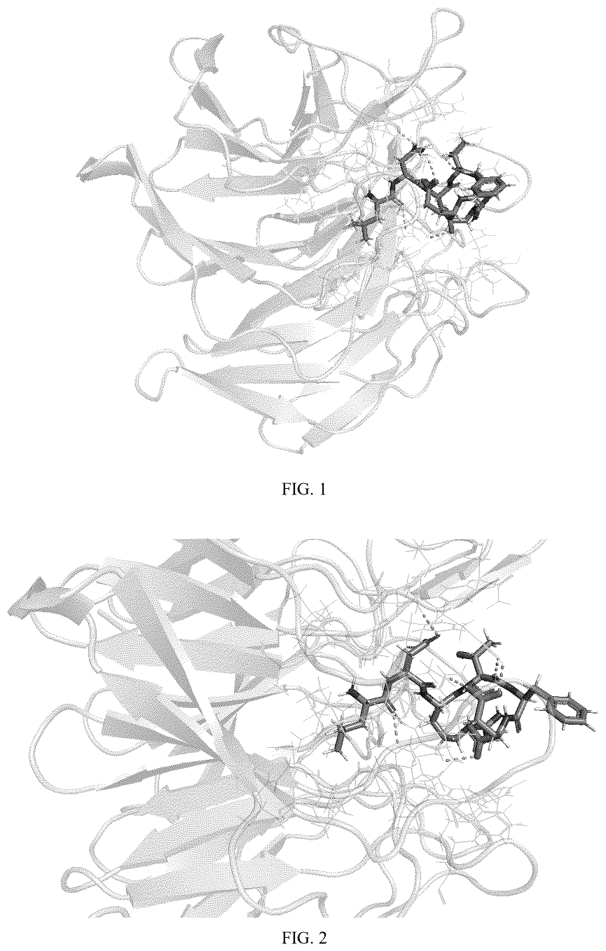

Through analysis, the binding mode between the hexapeptide LNRTFE (SEQ ID NO: 1) and Keap1 is shown in FIG. 1 and FIG. 2, and the details of the molecular docking are as follows:

Five H—H bond interactions, three C—H bond interactions, one salt bridge interaction, and two electrostatic interactions were formed between the hexapeptide LNRTFE (SEQ ID NO: 1) and Keap1, with five amino acid residues involved in the interaction between the hexapeptide LNRTFE (SEQ ID NO: 1) and Keap1.

### VI. Evaluation of the Antioxidant Function of Hexapeptide LNRTFE (SEQ ID NO: 1)

### 1. Solid-Phase Synthesis of Hexapeptide LNRTFE (SEQ ID NO: 1)

The Fmoc solid-phase synthesis strategy was adopted, with Fmoc-protected amino acids as a raw materials and polystyrene resin selected as the solid-phase carrier to conduct the solid-phase synthesis of the hexapeptide LNRTFE (i.e., PCT-1, SEQ ID NO: 1).

### 2. Animal Grouping and Bioactive Peptide Treatment

Positive control: 6-hydroxy-2,5,7,8-tetramethylchroman-2-carboxylic acid (Trolox).

Male ICR mice aged 8 weeks were selected, and reared under the conditions of a temperature of 20-26° C., air humidity of 50%-60%, and a 12-hour light-dark cycle, with free access to food and water. After 7 days of adaptive feeding, the mice were randomly divided into groups (control group, Trolox group, and PCT-1 group), with 12 mice in each group. Specifically, the control group was gavaged once a day with normal saline; the Trolox group was gavaged once a day with Trolox (dissolved in normal saline at a concentration of 1 mg/mL); the PCT-1 group was gavaged once a day with the solid-phase synthesized PCT-1 (dissolved in normal saline at a concentration of 1 mg/mL). The gavage volume was 100 μL per 10 g of body weight, and the gavage was continued for 30 days. During the experiment, all groups had free access to food and water.

### 3. Detection of the Effect of PCT-1 Treatment on Physiological Indicators and Serum Antioxidant Indicators in Mice

At the end of the experimental cycle, all mice were anesthetized with enflurane. Blood was collected from the orbital plexus, followed by incubation in a 37° C. water bath for 10 min. The blood was then centrifuged at 3000 rpm and 4° C. for 15 min to separate the upper serum layer. The serum was aliquoted and stored in a −80° C. refrigerator for later use.

After the mice were sacrificed by cervical dislocation, their spleens, livers, and brains were quickly isolated. These organs were weighed and then stored in a −80° C. refrigerator for later use.

Corresponding kits were used to detect the activities of superoxide dismutase (SOD), catalase (CAT), and glutathione peroxidase (GSH-Px), as well as the content of malondialdehyde (MDA) in the serum.

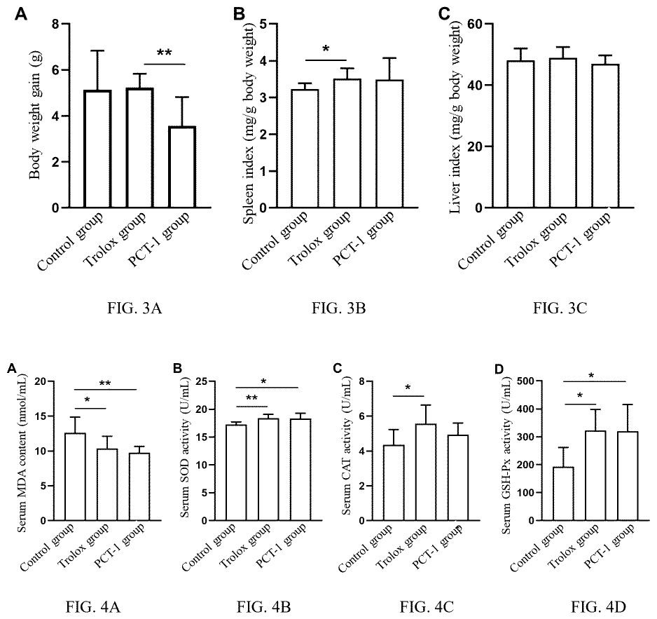

The effect of PCT-1 treatment on the physiological indicators (body weight gain, spleen index, liver index) of mice is shown in FIGS. 3A-3C, and its effect on the serum antioxidant indicators (MDA content, SOD activity, CAT activity, GSH-Px activity) of mice is shown in FIGS. 4A-4D.

As can be seen from FIGS. 3A-3C: Compared with the control group, the positive Trolox treatment significantly increased the spleen index of mice (p<0.05) but had no significant effect on body weight gain or liver index (p>0.05); PCT-1 treatment had no significant effect on body weight gain, spleen index, or liver index of mice (p>0.05). Compared with the positive Trolox treatment, PCT-1 treatment significantly reduced the body weight gain of mice (p<0.01) but had no significant effect on spleen index or liver index (p>0.05).

As can be seen from FIGS. 4A-4D: Compared with the control group, the positive Trolox treatment significantly increased the activities of SOD (p<0.01), CAT (p<0.05), and GSH-Px (p<0.05) in the serum, and significantly reduced the serum MDA content (p<0.05); PCT-1 treatment significantly increased the activities of SOD (p<0.05) and GSH-Px (p<0.05) in the serum, significantly reduced the serum MDA content (p<0.01), but had no effect on serum CAT activity (p>0.05). Compared with the positive Trolox treatment, PCT-1 treatment had no significant effect on serum MDA content, SOD activity, CAT activity, or GSH-Px activity in mice (p>0.05).

4. Quantitative Real-Time PCR (qRT-PCR) Detection

### (1) Primer Design for Quantitative Real-Time PCR

For the Keap1 protein, primers for quantitative real-time PCR (qRT-PCR) were designed using the NCBI website (https://www.ncbi.nlm.nih.gov/) and Primer 5 software from Premier (Canada). Details of the primers are shown in Table 3.

TABLE 3
Primer sequences for quantitative real-time PCR

| Gene | Forward primer | Reverse primer |
|---|---|---|
| Keap1 | TGGGTCAAATACGACTGCCC | TGGCTCATATCTCTCCACGC |
| | (SEQ ID NO: 26) | (SEQ ID NO: 27) |
| β-actin | CGCGAGTACAACCTTCTTGC | CGTCATCCATGGCGAACTGG |
| | (SEQ ID NO: 28) | (SEQ ID NO: 29) |

(2) Extraction of Total RNA from Tissues

100 mg each of mouse brain tissue and liver tissue was taken and placed in a sterile mortar pre-cooled with liquid nitrogen. Liquid nitrogen was added for rapid and thorough grinding, after which 1 mL of TrasZol Up reagent was added. The mixture was transferred to a DNase-free 1.5 mL centrifuge tube, and 0.2 mL of chloroform was added. The mixture was vortexed thoroughly for 30 s and allowed to stand for 10 min. Subsequently, centrifugation was performed at 12,000×g and 4° C. for 15 min, and the supernatant was transferred to another RNase-free 1.5 mL centrifuge tube. 0.5 mL of isopropanol was added, and the liquid in the tube was gently mixed, followed by standing at room temperature for 10 min. After that, centrifugation was performed again at 12,000×g and 4° C. for 10 min. The supernatant was discarded, and 1 mL of 75% ethanol (prepared with RNase-free water) was added to the precipitate to gently wash the precipitate. Centrifugation was then performed at 7,500×g and 4° C. for 5 min, and the supernatant was discarded. The centrifuge tube lid was opened to allow the ethanol to evaporate completely, after which 100 μL of RNase-free water was added. The concentration and quality of total RNA in the sample were quickly determined using a Nano Drop 2000c. The RNA was aliquoted into RNase-free centrifuge tubes and stored at −80° C.

(3) Synthesis of cDNA Template

1 μg of total RNA was taken, and cDNA was synthesized using the TransScript All-in-One First-Strand cDNA Synthesis SuperMix for qPCR (One-Step gDNA Removal) kit.

### (4) Detection of Gene Expression

Preparation of qRT-PCR system: After the cDNA template was appropriately diluted, 5 μL of it was added to the total system. 0.4 μL of forward primer and 0.4 μL of reverse primer were respectively added to the total system, followed by the addition of 10 μL of 2×TransStart™ Green qPCR SuperMix (containing SYBR Green dye) and 4.2 μL of nuclease-free water to the total system. The total volume of the system was 20 μL.

Three-step qRT-PCR protocol: 94° C. for denaturation 10 min; amplification reaction: 40 cycles of 94° C. for denaturation 15 s, 50° C. for annealing 15 s, and 72° C. for extension 45 s; 72° C. for extension 10 min.

The effect of PCT-1 treatment on the transcriptional level of the keap1 gene in the brain and liver of mice is shown in FIGS. 5A-5B.

As can be seen from FIGS. 5A-5B: Compared with the control group, the transcriptional level of the keap1 gene in the brain and liver was significantly downregulated by both the positive Trolox treatment and PCT-1 treatment, while no significant difference was observed between the positive Trolox treatment and PCT-1 treatment.

### VII. Evaluation of the Intestinal Flora-Regulating Function of Hexapeptide LNRTFE (SEQ ID NO: 1)

### 1. Animal Grouping and Bioactive Peptide Treatment

Male ICR mice aged 8 weeks were selected and reared under the conditions of a temperature of 20-26° C., air humidity of 50%-60%, a 12-hour light-dark cycle, and free access to food and water. After 7 days of adaptive feeding, the mice were randomly divided into groups (control group and PCT-1 group), with 12 mice in each group. Specifically, the control group was gavaged once a day with normal saline, while the PCT-1 group was gavaged once a day with the previously solid-phase synthesized PCT-1 (dissolved in normal saline at a concentration of 1 mg/mL). The gavage volume was 100 μL per 10 g of body weight, and the gavage was continued for 30 days. During the experiment, all groups had free access to food and water.

### 2. Detection of the Effect of PCT-1 Treatment on Intestinal Flora in Mice

At the end of the experimental cycle, mouse feces were collected and stored in a −80° C. refrigerator for later use.

### (1) Flora Sequencing in Mouse Feces

Total DNA was extracted from mouse feces using the QIAamp DNA Stool Mini Extraction Kit and stored in a −20° C. refrigerator for later use.

The DNA concentration was determined using a Thermo NanoDrop 2000 UV-Vis spectrophotometer.

PCR primers were designed based on the hypervariable region of the 16S rRNA gene. The nucleotide sequence of the forward primer was 5′-ACTCCTACGGGAGGCAGCAG-3′ (SEQ ID NO: 30), and the nucleotide sequence of the reverse primer was 5′-GGACTACHVGGGTWTCTAAT-3′ (SEQ ID NO: 31).

PCR amplification was performed, with a total reaction volume of 25 μL. The components of the PCR amplification system were as follows: 2 μL of genomic DNA (10 ng/μL), 2 μL of forward primer (0.1 μM), 2 μL of reverse primer (0.1 μM), 12.5 μL of premix, and ddH₂O to make up the total volume to 25 μL. The PCR amplification program was as follows: denaturation at 95° C. for 5 min; 20 cycles of denaturation at 95° C. for 30 s, annealing at 54° C. for 30 s, and extension at 72° C. for 40 s; final extension at 72° C. for 5 min.

After amplification, the PCR products obtained from PCR amplification were detected by agarose gel electrophoresis, followed by gel extraction and purification. After accurate quantification, the products were sequenced on the Illumina MiSeq platform.

The raw FASTQ files were processed using QIIME 1.8.0 as follows:
(i) Paired reads were assembled into a single sequence using FLASH 1.2.11 based on the overlap between PE reads, and sequences that could not be merged were removed;
(ii) After identifying the sample source of the data, the barcodes were removed;
(iii) Fragments shorter than 50 bp and bases with a quality score below 20 at the end of reads were removed using Prinseq software; finally, low-complexity sequences were filtered out, and non-amplified regions were removed;
(iv) The cluster algorithm (Mothur 1.36.0) was used for sequencing error correction, and chimeras in the sequences were removed simultaneously (Uchime 4.2.40).

The α-diversity of intestinal flora was analyzed using Mothur 1.36.0, and principal coordinate analysis was completed using Muscle 3.3.31.

The processed sequences were subjected to species annotation and community change analysis using the RDP Classifier software.

The effect of PCT-1 treatment on the α-diversity of intestinal flora is shown in Table 4.

TABLE 4
Effect of PCT-1 treatment on the α-Diversity of intestinal flora

| | ACE | Chao | Shannon | Simpson |
|---|---|---|---|---|
| | index | index | index | index |
| Control | 688.6 ± 184.08 | 677.4 ± 178.72 | 4.61 ± 0.67 | 0.046 ± 0.075 |
| group | | | | |
| PCT-1 | 596.2 ± 145.10 | 588.9 ± 141.79 | 4.49 ± 0.52 | 0.038 ± 0.026 |
| group | | | | |

As can be seen from Table 4: Compared with the control group, PCT-1 treatment reduced the ACE index, Chao index, Shannon index, and Simpson index of the intestinal flora in mice.

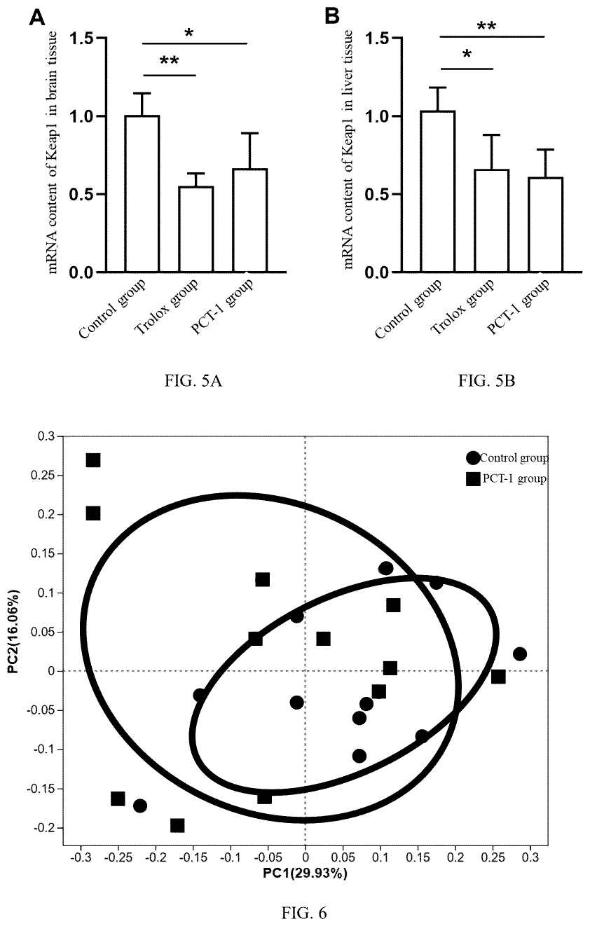

The effect of PCT-1 treatment on the overall composition of mouse intestinal flora was analyzed by weighted principal coordinate analysis. The analysis results are shown in FIG. 6.

As can be seen from FIG. 6: PCT-1 treatment altered the overall structure (β-diversity) of the intestinal flora in mice to a certain extent.

At the phylum level, the effect of PCT-1 treatment on the composition of mouse intestinal flora is shown in Table 5.

TABLE 5
Effect of PCT-1 treatment on the composition
of mouse intestinal flora at the phylum level

| | Control group | PCT-1 group |
|---|---|---|
| Bacteroidota | 0.454 ± 0.095 | 0.434 ± 0.132 |
| Firmicutes | 0.449 ± 0.105 | 0.447 ± 0.105 |
| Campilobacterota | 0.046 ± 0.049 | 0.044 ± 0.056 |
| Actinobacteriota | 0.025 ± 0.034 | 0.048 ± 0.052 |
| Patescibacteria | 0.013 ± 0.007 | 0.016 ± 0.009 |
| Desulfobacterota | 0.009 ± 0.012 | 0.008 ± 0.008 |
| Other | 0.005 ± 0.002 | 0.003 ± 0.002 |

As can be seen from Table 5: Compared with the control group, the PCT-1 treatment increased the abundance of Actinobacteriota and Patescibacteria in the intestinal tract of mice, while decreasing the abundance of Bacteroidota, Firmicutes, Campilobacterota, and Desulfobacterota. It is evident that the PCT-1 treatment has an effect on the composition of the intestinal flora in mice at the phylum level.

At the family level, the effect of the PCT-1 treatment on the composition of the intestinal flora in mice is shown in Table 6.

TABLE 6
Effect of PCT-1 treatment on the composition
of mouse intestinal flora at the family level

| | Control group | PCT-1 group |
|---|---|---|
| Muribaculaceae | 0.33 ± 0.096 | 0.336 ± 0.126 |
| Lactobacilluseae | 0.135 ± 0.143 | 0.138 ± 0.098 |
| Lachnospiraceae | 0.112 ± 0.068 | 0.11 ± 0.081 |
| Bacillaceae | 0.056 ± 0.049 | 0.045 ± 0.037 |
| norank_o_{——}Clostridia_UCG-014 | 0.054 ± 0.033 | 0.045 ± 0.029 |
| Bacteroidaceae | 0.042 ± 0.023 | 0.03 ± 0.028 |
| Helicobacteraceae | 0.046 ± 0.049 | 0.044 ± 0.056 |
| Prevotellaceae | 0.037 ± 0.031 | 0.039 ± 0.057 |
| Erysipelotrichaceae | 0.019 ± 0.033 | 0.044 ± 0.046 |
| Bifidobacteriaceae | 0.019 ± 0.033 | 0.04 ± 0.046 |
| Other | 0.15 ± 0.057 | 0.131 ± 0.046 |

As can be seen from Table 6: Compared with the control group, the PCT-1 treatment increased the abundance of Muribaculaceae, Lactobacillus, Prevotellaceae, Erysipelotrichaceae, and Bifidobacteriaceae in the intestinal tract of mice, while decreasing the abundance of Lachnospiraceae, Bacillaceae, norank_o_Clostridia_UCG-014, Bacteroidaceae, and Helicobacteraceae. It is evident that the PCT-1 treatment also has an effect on the composition of the intestinal flora in mice at the family level.

The differentially expressed amplicon sequence variants (ASVs) in the intestinal flora after PCT-1 treatment were analyzed (p<0.05), and the analysis results are shown in Table 7.

TABLE 7
Analysis results of differentially expressed ASVs after PCT-1 treatment

| | Control | PCT-1 | |
|---|---|---|---|
| | group | group | Taxonomic level |
| ASV697 | 0.01685 | 0.3725 | Lactobacillus |
| ASV59 | 0.1783 | 0.1112 | norank_f_{——}Muribaculaceae |
| ASV87 | 0.01508 | 0.0609 | unclassified_f_{——}Lachnospiraceae |
| ASV217 | 0.1667 | 0.2081 | norank_f_{——}Muribaculaceae |
| ASV1033 | 0.1002 | 0.3119 | Lachnospiraceae_NK4A136_group |
| ASV148 | 0.3264 | 0.07598 | norank_f_{——}norank_o_{——}Clostridia_UCG-014 |
| ASV688 | 0 | 0.1212 | Prevotellaceae_UCG-001 |
| ASV1306 | 0.02129 | 0.1777 | unclassified_f_{——}Lachnospiraceae |
| ASV1035 | 0.007095 | 0.1055 | unclassified_f_{——}Lachnospiraceae |
| ASV1230 | 0.1109 | 0.01656 | Christensenellaceae_R-7_group |
| ASV7363 | 0.1387 | 0 | norank_f_{——}norank_o_{——}Clostridia_UCG-014 |
| ASV808 | 0.04612 | 0.002661 | norank_f_{——}Ruminococcaceae |
| ASV5520 | 0 | 0.003843 | norank_f_{——}Muribaculaceae |
| ASV1028 | 0.0612 | 0.03577 | Candidatus_Saccharimonas |
| ASV301 | 0.05647 | 0.01005 | Butyricicoccus |
| ASV1024 | 0 | 0.08722 | Lactobacillus |
| ASV182 | 0.05085 | 0.02158 | unclassified_f_{——}Ruminococcaceae |
| ASV132 | 0.0133 | 0.05676 | norank_f_{——}norank_o_{——}Clostridia_UCG-014 |
| ASV1972 | 0.05972 | 0 | unclassified_f_{——}Lachnospiraceae |
| ASV1950 | 0.0337 | 0.006504 | norank_f_{——}Ruminococcaceae |
| ASV2795 | 0.005617 | 0 | Candidatus_Saccharimonas |
| ASV5525 | 0 | 0 | norank_f_{——}Erysipelotrichaceae |
| ASV254 | 0.02395 | 0.01744 | Lachnospiraceae_FCS020_group |
| ASV2410 | 0 | 0.03991 | norank_f_{——}Muribaculaceae |
| ASV3379 | 0 | 0.03903 | unclassified_f_{——}Lachnospiraceae |
| ASV2016 | 0.002956 | 0.007391 | norank_f_{——}norank_o_{——}Clostridia_vadinBB60_group |
| ASV3634 | 0.00207 | 0.03636 | norank_f_{——}norank_o_{——}Clostridia_UCG-014 |
| ASV2815 | 0.01626 | 0 | norank_f_{——}norank_o_{——}Clostridia_UCG-014 |
| ASV1550 | 0.0272 | 0 | unclassified_o_{——}Oscillospirales |
| ASV5524 | 0 | 0 | Lachnospiraceae_UCG-001 |
| ASV776 | 0.02365 | 0.007095 | unclassified_f_{——}Ruminococcaceae |
| ASV1915 | 0.03075 | 0.00207 | Bacteroides |
| ASV181 | 0.007982 | 0.01951 | unclassified_f_{——}Oscillospiraceae |
| ASV2253 | 0.006504 | 0.01685 | norank_f_{——}Lachnospiraceae |
| ASV1538 | 0.02572 | 0 | Erysipelatoclostridium |
| ASV1101 | 0 | 0.01035 | norank_f_{——}norank_o_{——}Clostridia_UCG-014 |
| ASV344 | 0.00207 | 0.01774 | Acetatifactor |
| ASV221 | 0 | 0.0201 | Colidextribacter |
| ASV5539 | 0 | 0 | norank_f_{——}Muribaculaceae |
| ASV5530 | 0 | 0 | norank_f_{——}Muribaculaceae |
| ASV2205 | 0 | 0.01626 | unclassified_f_{——}Lachnospiraceae |
| ASV1299 | 0.01478 | 0 | ASF356 |
| ASV1961 | 0.01301 | 0 | unclassified_f_{——}Oscillospiraceae |
| ASV288 | 0 | 0.01242 | norank_f_{——}Ruminococcaceae |
| ASV7832 | 0 | 0.01212 | Colidextribacter |
| ASV10495 | 0.01094 | 0 | norank_f_{——}norank_o_{——}Clostridia_UCG-014 |
| ASV372 | 0.001478 | 0.009165 | norank_f_{——}norank_o_{——}Clostridia_UCG-014 |
| ASV6162 | 0 | 0 | norank_f_{——}norank_o_{——}Clostridia_vadinBB60_group |
| ASV2829 | 0 | 0 | norank_f_{——}Muribaculaceae |
| ASV2192 | 0 | 0.008574 | Christensenellaceae_R-7_group |
| ASV5561 | 0 | 0 | norank_f_{——}norank_o_{——}Clostridia_UCG-014 |
| ASV4676 | 0.006504 | 0.0008869 | unclassified_f_{——}Lachnospiraceae |
| ASV1661 | 0.006504 | 0 | norank_f_{——}UCG-010 |
| ASV9735 | 0 | 0 | norank_f_{——}norank_o_{——}RF39 |
| ASV1767 | 0.004139 | 0 | norank_f_{——}norank_o_{——}Clostridia_UCG-014 |
| ASV6191 | 0 | 0 | norank_f_{——}norank_o_{——}Clostridia_vadinBB60_group |
| ASV7250 | 0.003252 | 0 | norank_f_{——}norank_o_{——}RF39 |
| ASV416 | 0 | 0.002956 | ASF356 |

As can be seen from Table 7: Compared with the control group, the PCT-1 treatment upregulated the abundance of 25 ASVs and downregulated the abundance of 24 ASVs. It is evident that the PCT-1 treatment also has an effect on the expression of ASVs.

### (2) Analysis of Short-Chain Fatty Acid Content in Mouse Feces

30 mg of dried mouse feces was weighed, and 0.8 mL of water was added. After shaking to mix well, 0.2 mL of sulfuric acid with a mass concentration of 50% was added. Following uniform mixing, 1 mL of diethyl ether was added, and the mixture was thoroughly mixed for 30 min, then centrifuged at 10,000 rpm for 10 min. Calcium chloride was added to the supernatant to absorb water, and the mixture was filtered through a 0.22 μm filter membrane. 1 mL of the filtrate was taken for sample loading. Using the external standard method, standard curves for acetic acid, propionic acid, and butyric acid were plotted respectively. A gas chromatography-mass spectrometry (GC-MS) system (Agilent 7890 Gas Chromatograph coupled with Puxi M7-80E Mass Spectrometer) was adopted, with a flame ionization detector (FID) and a DB-WAX chromatographic column (30 m×0.25 mm×0.25 μm). Helium was used as the carrier gas, with a split ratio of 20:1 and a flow rate of 1 mL/min, and the injection volume was 1 μL. The initial temperature of the column oven was set to 90° C., which was increased to 150° C. at a rate of 12° C./min, then further increased to 220° C. at a rate of 20° C./min, and maintained at 220° C. for 4.5 min. The temperatures of the transfer line and ion source were set to 220° C. and 230° C. respectively, and the solvent delay time was set to 3 min.

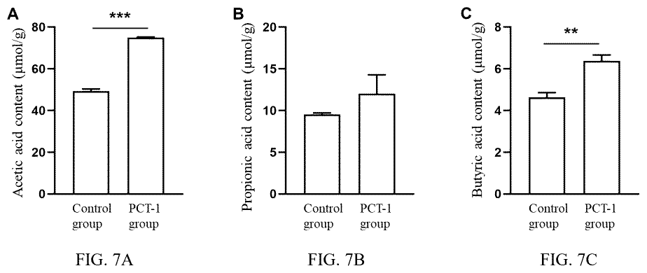

The detection results of the contents of acetic acid, propionic acid, and butyric acid in the intestinal tract of mice after PCT-1 treatment are shown in FIGS. 7A-7C.

As can be seen from FIGS. 7A-7C: Compared with the control group, the PCT-1 treatment significantly increased the content of acetic acid (p<0.001) and butyric acid (p<0.01) in mouse feces, but had no effect on the content of propionic acid.

In conclusion, the hexapeptide LNRTFE (PCT-1, SEQ ID NO: 1) screened from spirulina protein peptides in the present invention has antioxidant and intestinal flora-regulating functions, and can be applied in functional foods for antioxidation and intestinal flora regulation.

It should be noted that the above examples are only for clearly illustrating the present invention, and are not intended to limit the embodiments of the present invention. For those of ordinary skill in the art, on the basis of the above description, various other forms of changes or modifications can also be made. It is impossible to enumerate all embodiments here. All obvious changes or modifications derived from the technical solution of the present invention still fall within the protection scope of the present invention.

What is claimed is:

1. A bioactive peptide PCT-1, wherein the amino acid sequence of the bioactive peptide PCT-1 is LNRTFE, as shown in SEQ ID NO: 1 in the sequence listing, and the bioactive peptide PCT-1 has antioxidant and intestinal flora-regulating functions.

2. An application of the bioactive peptide PCT-1 according to claim 1 in functional foods for antioxidation and intestinal flora regulation.

3. A preparation method of the bioactive peptide PCT-1 according to claim 1, wherein the preparation method adopts a solid-phase synthesis method, specifically:
using Fmoc-protected amino acids as raw materials and polystyrene resin as a solid-phase carrier, and performing solid-phase synthesis by adopting an Fmoc solid-phase synthesis strategy.

4. A preparation method of the bioactive peptide PCT-1 according to claim 1, wherein the preparation method adopts an enzymatic hydrolysis method, specifically:
(1) taking spirulina protein, adding water with a mass 9 times that of the spirulina protein, and adding a compound protease at an enzyme-to-substrate ratio of 0.5% for enzymatic hydrolysis; conducting the enzymatic hydrolysis at a temperature of 55° C. and a pH value of 8.0 for 6 h to obtain an enzymatic hydrolysate, wherein the compound protease is formed by mixing alkaline protease and neutral protease at a mass ratio of 1:1;
(2) after the enzymatic hydrolysis is completed, filtering the enzymatic hydrolysate with eight layers of gauze to remove residues, so as to obtain a spirulina protein peptide enzymatic hydrolysate; and
(3) freeze-drying the spirulina protein peptide enzymatic hydrolysate to obtain a spirulina protein peptide powder, wherein the spirulina protein peptide powder contains a relatively large amount of the bioactive peptide PCT-1.
